(19) United States
(12) Patent Application Publication
(10) Pub. No.: US 2021/0142871 A1
(43) Pub. Date: May 13, 2021

(54) METHOD FOR DETERMINING EFFECT FOR AN INDIVIDUAL PATIENT

(71) Applicant: Innoplexus AG, Eschborn (DE)

(72) Inventors: Nitish Jain, Jaipur (IN); Ratan Dua, Bhadohi (IN); Vipul Patni, Shrirampur (IN)

(21) Appl. No.: 17/092,431

(22) Filed: Nov. 9, 2020

(30) Foreign Application Priority Data
Nov. 8, 2019 (IN) 201921045514

(51) Int. Cl. G16H 10/20; G06F 16/242; G06F 7/552

(52) U.S. Cl. CPC G16H 10/20; G06F 7/552; G06F 16/242

(57) ABSTRACT

Disclosed is system and method for determining treatment effect in a clinical trial, the system comprising a server arrangement communicably coupled to a database arrangement. The server arrangement is configured to receive a plurality of datasets corresponding to a plurality of subjects from the database arrangement, wherein the plurality of subjects comprises a first set of subjects undergoing treatment and a second set of subjects receiving placebo, and wherein the plurality of datasets comprises of plurality independent variables and at least one dependent variables for the plurality of subjects. The system is configured to impute one or more values in the plurality of datasets to generate an imputed dataset of the plurality of subjects, wherein the imputed dataset comprises of imputed and non-imputed values corresponding to the plurality of independent variables and the at least one dependent variable. The system is further configured to normalize the imputed dataset corresponding to the plurality of independent variable of the plurality of subjects, determine a plurality of weightage score of the plurality of independent variables with respect to the at least one dependent variable of the second set of subjects, determine a plurality of similarity score between each of the first set of subjects undergoing treatment to each of the second set of subjects receiving placebo, determine a placebo effect for each of the first set of subjects undergoing treatment, and determine a treatment effect for each of the first set of subjects undergoing treatment.

### CROSS REFERENCE TO RELATED APPLICATIONS

This application claims priority to Indian Provisional Patent Application No. 201921045514 filed 8 Nov. 2019, which is hereby incorporated by reference in its entirety.

### TECHNICAL FIELD

The present disclosure relates generally to determination of treatment effect in clinical trial; and more specifically, to systems and methods for determining placebo effect in clinical trial.

### BACKGROUND

On the global stage, multitude of diseases claims millions of lives in a single day. With the rise in diseases leading to human sickness, healthy life expectancy is affected substantially. Therefore, development of new drugs for present non-curable diseases, reliable and improved cure of curable diseases and/or economical alternate for presently deployed drug, is vital to sustain healthy human life expectancy.

It will be appreciated that development of new drugs is a long, complex and costly process. Moreover, studies have shown that majority of newly developed drugs, when tested on human subjects, fail in clinical trials. Therefore, clinical trials play a crucial role in establishing therapeutic response for newly developed drugs. It will be appreciated that clinical trial is an essential step in a drug discovery process in order to establish a therapeutic response for human subjects and further get approval from a regulatory body for commercialization of the drug.

In a clinical trial, a large number of human subjects are involved, wherein a first set of human subjects (namely, a treatment arm) are subjected to a drug and a second set of human subjects (namely, a placebo arm) are subjected to placebo treatment. Furthermore, for a human subject from the treatment arm, a total effect of the drug is a summation of drug effect and placebo effect. Additionally, for a human subject from the placebo arm, a total effect of the drug is placebo effect. Subsequently, the total effect of the human subject from the treatment arm and the human subject from the placebo arm are processed to determine the drug effect on the human subjects in treatment arm.

However, such determination of drug effect on human subjects is prone to errors owing to large number of discrepancies, missing information and varied background information associated with human subjects participating in clinical trials. Additionally, division of the human subjects participating in the clinical trial into treatment arm and placebo arm, based on background information associated therewith, is not homogenous. The division of the human subjects into the treatment arm and the placebo arm is further affected in case of small number of human subjects participating in the clinical trial. Subsequently, results of the clinical trial are prone to errors. Moreover, such inaccurate results of clinical trials may depreciate an effective drug or appreciate an ineffective drug. Additionally, drug discovery process is delayed owing to inaccurate results of the clinical trial thereby increasing a development cost thereof. Such costs associated with the drug contribute to commercial value thereof thereby making the drug uneconomical.

Therefore, in light of foregoing discussion, there exists a need to overcome aforesaid drawbacks associated with conventional process of implementing clinical trials for drug discovery.

### SUMMARY

The present disclosure seeks to provide a system for determining treatment effect of a subject in clinical trial. The present disclosure also seeks to provide a method for determining treatment effect of a subject in clinical trial. The present disclosure seeks to provide a solution to the existing problem of unreliable methods of calculating treatment effect of a subject in clinical trial. An aim of the present disclosure is to provide a solution that overcomes at least partially the problems encountered in prior art and provides a system that accurately determines treatment effect of the subject in clinical trial.

In one aspect, an embodiment of the present disclosure provides a system for determining treatment effect in a clinical trial, the system comprising a server arrangement communicably coupled to a database arrangement, wherein the server arrangement is configured to:

(a) receive a plurality of datasets corresponding to a plurality of subjects from the database arrangement, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) impute one or more values in the plurality of datasets to generate an imputed dataset of the plurality of subjects, wherein the imputed dataset comprises of imputed and non-imputed values corresponding to the plurality of independent variables and the at least one dependent variable,
(c) normalize the imputed dataset corresponding to the plurality of independent variable of the plurality of subjects,
(d) determine a plurality of weightage score of the plurality of independent variables with respect to the at least one dependent variable of the second set of subjects,
(e) determine a plurality of similarity score between each of the first set of subjects undergoing treatment to each of the second set of subjects receiving placebo,
(f) determine a placebo effect for each of the first set of subjects undergoing treatment, and
(g) determine a treatment effect for each of the first set of subjects undergoing treatment.

In another aspect, an embodiment of the present disclosure provides a method of determining treatment effect of a clinical trial, wherein the method comprises:

(a) receiving a plurality of datasets corresponding to a plurality of subjects, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) imputing one or more values in the plurality of datasets to generate an imputed dataset of the plurality of subjects, wherein the imputed dataset comprises of imputed and non-imputed values corresponding to the plurality of independent variables and the at least one dependent variable,
(c) normalizing the imputed dataset corresponding to the plurality of independent variable of the plurality of subjects,
(d) determining a plurality of weightage score of the plurality of independent variables with respect to the at least one dependent variable of the second set of subjects,
(e) determining a plurality of similarity scores between each of the first set of subjects undergoing treatment to each of the second set of subjects receiving placebo,
(f) determining a placebo effect for each of the first set of subjects undergoing treatment, and
(g) determining a treatment effect for each of the first set of subjects undergoing treatment.

In yet another aspect, an embodiment of the present disclosure provides a computer program product comprising non-transitory computer-readable storage media having computer-readable instructions stored thereon, the computer-readable instructions being executable by a computerized device comprising processing hardware to execute the aforesaid method.

Embodiments of the present disclosure substantially eliminate or at least partially address the aforementioned problems in the prior art and enables automated estimation of treatment effect of the subject during clinical trial thereby removing any human bias in calculation of such treatment effect.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a data flow diagram representing a system for determining treatment effect in a clinical trial.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a system for determining treatment effect in a clinical trial, the system comprising a server arrangement communicably coupled to a database arrangement, wherein the server arrangement is configured to:

(a) receive a plurality of datasets corresponding to a plurality of subjects from the database arrangement, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) impute one or more values in the plurality of datasets to generate an imputed dataset of the plurality of subjects, wherein the imputed dataset comprises of imputed and non-imputed values corresponding to the plurality of independent variables and the at least one dependent variable,
(c) normalize the imputed dataset corresponding to the plurality of independent variable of the plurality of subjects,
(d) determine a plurality of weightage score of the plurality of independent variables with respect to the at least one dependent variable of the second set of subjects,
(e) determine a plurality of similarity score between each of the first set of subjects undergoing treatment to each of the second set of subjects receiving placebo,
(f) determine a placebo effect for each of the first set of subjects undergoing treatment, and
(g) determine a treatment effect for each of the first set of subjects undergoing treatment.

In another aspect, an embodiment of the present disclosure provides a method of determining treatment effect of a clinical trial, wherein the method comprises:

(a) receiving a plurality of datasets corresponding to a plurality of subjects, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) imputing one or more values in the plurality of datasets to generate an imputed dataset of the plurality of subjects, wherein the imputed dataset comprises of imputed and non-imputed values corresponding to the plurality of independent variables and the at least one dependent variable,
(c) normalizing the imputed dataset corresponding to the plurality of independent variable of the plurality of subjects,
(d) determining a plurality of weightage score of the plurality of independent variables with respect to the at least one dependent variable of the second set of subjects,
(e) determining a plurality of similarity scores between each of the first set of subjects undergoing treatment to each of the second set of subjects receiving placebo,
(f) determining a placebo effect for each of the first set of subjects undergoing treatment, and
(g) determining a treatment effect for each of the first set of subjects undergoing treatment.

The system for determining treatment effect of the clinical trial enables accurate determination of treatment effect of the clinical trial in an automated manner. The system is of advantage as it enables determination of treatment effect of a clinical trial comprising small number of participants (namely, subjects), wherein homogenous distribution of the subjects into treatment arm and placebo is not viable. Therefore, the system enables implementation of a clinical trial with lesser number of subjects thereby substantially reducing cost and manpower required for implementation of the clinical trial. Moreover, the system reliably determines the treatment effect of the subject during clinical trials where the subjects are not distributed homogenously.

The present disclosure discloses the system for determining treatment effect of the clinical trial. It will be appreciated that clinical trials are scientific studies (including tests, experiments and observations) conducted to establish new interventions, treatments or tests as a means to prevent, screen, diagnose, or treat diseases or medical conditions. These clinical trials are conducted on subjects, specifically, human subjects. Furthermore, such scientific studies are performed to obtain specific information related to biomedical or behavioral interventions, including new treatments (such as, novel vaccines, drugs, dietary choices, dietary supplements and medical devices and so forth) and known interventions that require further study and comparison. Additionally, a clinical trial is carried out in a number of phases involving different constraints applied for conducting the clinical trial. Moreover, the clinical trial may have a number of versions depending upon date of the clinical trial. Furthermore, clinical trials for a specific drug may be conducted in different geographical locations and under varying environmental conditions. Such clinical trials may be provided to an approving body in order to validate authentication of the clinical trial and approve use thereof by the public. Notably, the clinical trials are conducted in a randomized, double blinded, and placebo-controlled clinical trial having one or multiple treatment groups and a placebo treatment groups. In an example, in a double-blinded trial, the placebo effect across groups remains the same for patients with similar physical and social characteristics, environment, patient, doctor or assessor interaction. In order to determine potency or treatment effect an intervention (drug or therapy) on the patients in treatment group of this type of clinical trial, the invention determines the similarity of each treatment group patient with all the placebo group patients based on various independent patient characteristics and trial design features to predict the contribution of placebo effect to the total effect of the intervention.

Notably, the “server arrangement” refers to a structure and/or module that include programmable and/or non-programmable components configured to store, process and/or share information. The server is a computational element that is operable to respond to and processes instructions that drive the system. Optionally, the server arrangement includes any arrangement of physical or virtual computational entities capable of enhancing information to perform various computational tasks. Furthermore, it should be appreciated that the server arrangement may be both single hardware server and/or plurality of hardware servers operating in a parallel or distributed architecture. In an example, the server arrangement may include components such as memory, a processor, a network adapter and the like, to store, process and/or share information with other computing components, such as the user device. Optionally, the server arrangement is implemented as a computer program that provides various services (such as database service, computing power service, and the like) to other devices, modules or apparatus.

The system comprise the server arrangement communicably coupled to the at least one user device. The “user device” refers to an electronic device associated with (or used by) a user that is capable of enabling the user to perform specific tasks associated with the aforementioned system. Furthermore, the user device is intended to be broadly interpreted to include any electronic device that may be used for voice and/or data communication over a wireless communication network. Examples of user device include, but are not limited to, cellular phones, personal digital assistants (PDAs), handheld devices, wireless modems, laptop computers, personal computers, etc. Additionally, optionally, the user device includes a casing, a memory, a processor, a network interface card, a microphone, a speaker, a keypad, and a display.

The server arrangement is configured to receive the plurality of datasets corresponding to the plurality of subjects from a database arrangement. The plurality of subjects comprises a first set of subjects undergoing treatment and a second set of subjects receiving placebo. Moreover, each of the plurality of datasets include values relating to the plurality of variables, wherein the plurality of variables comprises of plurality of independent variables and at least one dependent variable. Notably, the plurality of subjects refers to participants of the clinical trial. It will be appreciated that the plurality of subjects includes patients having one or more medical condition and/or undergoing a medical treatment, healthy subjects having no medical condition and/or not undergoing any medical treatment, or a combination thereof.

Pursuant to embodiments of the present disclosure, the server arrangement receives the plurality of subject datasets from, for example, a database associated with the server arrangement, a user device associated with a subject from the plurality of subjects, a third-party database, or a combination thereof. It will be appreciated that a database storing the plurality of subject datasets refer to an organized body of digital information regardless of the manner in which data (the plurality of subject datasets) or the organized body thereof is represented. Optionally, the database may be hardware, software, firmware and/or any combination thereof. More optionally, the plurality of subject datasets in the database may be in the form of a table, a map, a grid, a packet, a datagram, a file, a document, a list, and the like. The database includes any data storage software and systems, such as, for example, a relational database like IBM DB2 and Oracle 9. Optionally, the database is operable to support relational operations, regardless of whether it enforces strict adherence to the relational model, as understood by those of ordinary skill in the art. Additionally, the database is populated by data elements (namely, the plurality of subject datasets), wherein the data elements may be include data records, bits of data, cells, and the like and all intended to mean information stored in cells of a database.

It will be appreciated that each of the plurality of subjects have a subject dataset associated thereto. Moreover, a subject dataset associated with a subject from the plurality of subjects comprise subject values corresponding to the plurality of subject parameters, associated with the subject. In an example, the subject dataset is a document, for example, a (.doc) text file, an excel (.xls) file, an image (.jpg, or .png) file, and the like.

Herein, the plurality of subjects comprises of the first set of subjects undergoing treatment and the second set of subjects receiving placebo. In other words, the plurality of subjects is divided into two groups, namely, the first set of subjects and the second set of subjects. Moreover, it will be appreciated that the first set of subjects undergoing treatment receives a drug or therapy as per the clinical trial. The second set of subjects receiving placebo which include inert drugs or does not contain active ingredients and is made to be physically indistinguishable from the actual drug being studied. It will be appreciated that the placebo comprise treatment of the second set of subjects without using an active substance that affect health of the second set of subjects.

Furthermore, the plurality of datasets comprises of plurality independent variables and at least one dependent variables for the plurality of subjects. The plurality of datasets includes plurality of values associated with a corresponding subject. Optionally, the plurality of datasets may be recorded, collected or stored previously by a user of the system, by corresponding subject, by a third party, or a combination thereof. Additionally, optionally, information associated with the plurality of subjects are recorded in the corresponding plurality of datasets in form of, for example, tables, charts, graphs, pictures, and so forth. Optionally, the plurality of independent variables includes independent variables related to patient characteristics and clinical trial. In an embodiment, the plurality of independent variables related to patient characteristics include, demographics, genetic profile of the patients, disease or indication details, baseline test scores or scales capturing the disability or damage or deformity due to the disease or indication, baseline vital signs, medical history, concomitant medications, treatment emergent adverse events. The demographics include age, gender, race, ethnicity, height, weight, BMI etc. The disease or indication details includes intensity, severity, location, time of occurrence, age at the time of occurrence, age since occurrence. The baseline vital signs include systolic BP, diastolic BP, respiratory rate, heart rate, temperature. The treatment emergent adverse events include count, severity and seriousness. In an embodiment, the plurality of independent variables related to clinical trial includes dosage, surgery site, assessment site, type of equipment used for the treatment, per protocol flag, completed flag, and protocol deviations. Optionally, the at least one dependent variable is based on improvement over baseline on an index measured at pre-defined frequency during the clinical trial. In an example, a clinical trial for a weight loss may include the above-mentioned independent variables, wherein the subject has been able to reduce his/her weight from 100 KG to 90 KG in 3 months, subsequently the measured weight of the subject after 6 months was 80 KG. In an embodiment, the value associated with at least one dependent variable is 20 points after 6 months i.e. based on the improvement over baseline on an index measured at pre-defined frequency during the clinical trial.

Optionally, the independent variables comprise of continuous variables and categorical variables. Typically, the variables for example, age, weight, vital signs associated with the baseline parameters, and the like having numerical (quantitative) subject values form continuous subject parameters. The variables for example, race, ethnicity, gender, genetic parameters, and the like having certain specific subject value (qualitative) are categorical subject parameters. In an example, the age of a subject is 32 years, whereas the gender is Female.

The system is configured to impute the one or more values in the plurality of datasets to generate an imputed dataset of the plurality of subjects. The imputed dataset comprises of imputed and non-imputed values corresponding to the plurality of independent variables and the at least one dependent variable. Beneficially, the imputing the one or more values in the plurality of dataset allow efficient operation of the system.

Optionally, the one or more values of the continuous variables of the independent variable are imputed by a mean of the values of the corresponding independent variable. Further optionally, the one or more values of the categorical variables of the independent variable is imputed by a mode of the values of the corresponding independent variable. In an example, for Subject 1, the value corresponding to ‘age’ is missing. The system is configured to impute the age of Subject 1 with the mean of all age values corresponding to all the subjects in clinical trial. In another example, for Subject 2, the value corresponding to ‘sex’ is missing. The system is configured to impute the value with the mode of all values corresponding to the value of ‘sex’ for all subjects. Furthermore optionally, the one or more values of the at least one dependent variable is imputed by Last Observed Carried Forward (LOCF) method of the corresponding dependent variable. In an example, the last known weight of the Subject 3 was 60 Kg, if any new observation has not been recorded then the system is configured to use Last Observed Carried Forward (LOCF) method.

The server arrangement is configured to normalize the imputed dataset corresponding to the plurality of the independent variables of the plurality of subjects. Beneficially, the system is configured to normalize the imputed dataset corresponding to the plurality of the independent variables of the first set of subjects and the second set of subjects.

Optionally, the continuous variables of the independent variable are normalized using Min-Max Scaler. Further optionally, the categorical variables of the independent variable are one-hot-encoded into dummy variables. Beneficially, machine learning Algorithms cannot work with categorical variables directly, they need to be converted to numbers. In an example, one-hot-encoder returns a value for each unique value of the categorical column. Each such vector contains only one ‘1’ while all other values in the vector are ‘0’. For example, gender variable takes categorical values of male or female. This variable is converted to two variables, namely gender_male and gender_female. For a male subject, gender_male takes value of ‘1’ and gender_female takes value of ‘0’ In another example, the continuous variables like age, weight, vital signs, etc. are normalized using Min-Max Scaler.

The server arrangement is configured to determine a plurality of weightage score of the plurality of independent variables with respect to the at least one dependent variable of the second set of subjects. Optionally, the weightage score is based on SHAP importance of XGBoost regressor model. In an embodiment, a decision tree regressor based model is used to measure how strongly does an independent variable (importance) explain the magnitude of Total Effect in the second set of subjects receiving placebo. The system is configured to process the second set of normalized imputed dataset of the plurality of independent variables and the least one dependent variable to determine the weightage score of the plurality of independent variables with respect to the at least one dependent variable. Optionally, an XGBoost Regressor model is trained using grid search for tuning the Hyperparameters of the said model. Then using the SHAP importance of the best estimator, the system determines important variables and their power to predict the Total Effect (ie. Placebo Effect) in the second set of subjects receiving placebo. An example to illustrate the weightage score for the plurality of independent variables is:

| Rank | Feature name | Weightage (lx) |
|---|---|---|
| 1 | Treatment Duration (Minutes) | 19.1% |
| 2 | Body Mass Index (Kg/m²) | 17.2% |
| 3 | Protocol Deviations - Tests/Assessments/ | 10.5% |
| | Procedures (Occurrences) | |
| 4 | Test Score 1 at Baseline Visit (Score) | 9.2% |
| 5 | Vital Signs - Systolic Blood Pressure at Baseline | 7.9% |
| | Visit (mmHg) | |
| 6 | Test Score 2 at Baseline Visit (Score) | 7.7% |
| 7 | Vital Signs - Temperature at Baseline Visit (° C.) | 7.2% |
| 8 | Test Score 3 at Baseline Visit (Score) | 5.8% |
| 9 | Weight (Kg) | 4.4% |
| 10 | Test Score 4 at Baseline Visit (Score) | 3.1% |
| 11 | Medical History_Surgical and medical procedures | 2.9% |
| | (Occurrences) | |
| 12 | Age (Years) | 2.7% |
| 13 | Medical History - Nervous system disorders | 2.5% |
| | (Occurrences) | |

Notably, the term “machine learning algorithms” refer to a category of algorithms employed by a processing device (herein, the server arrangement) implementing a software application. The machine learning algorithms allows the server arrangement to become more accurate in predicting weights and/or performing tasks associated with the system, without being explicitly programmed. Specifically, the machine learning algorithms are employed to artificially train the server arrangement to enable it to automatically learn, from analyzing training dataset and improving performance or output from experience, without being explicitly programmed, to efficiently execute the system.

Optionally, the machine learning algorithms, executed by the server arrangement, are trained using a training dataset. More optionally, the machine learning algorithms are trained using training dataset comprising labelled data, unlabeled data, or a combination thereof. In this regard, the machine learning algorithms undergo at least one of: unsupervised training, supervised training, reinforced training, semi-supervised training. Furthermore, the machine learning algorithms are trained by interpreting patterns in the training dataset and adjusting the machine learning algorithms accordingly to get a desired output.

Optionally, the server arrangement employs machine learning algorithms to determine the weightage score corresponding to each of the plurality of independent variables. Pursuant to embodiment of the present disclosure, the machine learning algorithms are trained in a supervised manner using labelled training dataset, i.e. the at least one dependent variable for the second set of subjects, to determine the weightage score for each of the plurality of independent variables.

The server arrangement is configured to determine a plurality of similarity score between each of the first set of subjects undergoing treatment to each of the second set of subjects receiving placebo. Optionally, the plurality of similarity score is based on an inverse of weighted Euclidean distance. Further optionally, the weighted Euclidean distance is calculated using the plurality of weightage score, the normalized imputed dataset of plurality of independent variables from first set of subjects and the normalized imputed dataset of plurality of independent variables from second set of subjects.

Optionally, the weighted Euclidean distance is calculated based on

Dᵢⱼ={square root over ((Iₓ(Xᵢ−Xⱼ))²+(I_{Y}(Yᵢ−Yⱼ))²+ . . . )}

where ‘i’ is the i'^{th} patient in the first set of subjects undergoing treatment and j is the j^{th} patient in the second set of subjects receiving placebo,
Dᵢⱼ is the weighted euclidean distance between these patients,
Iₓ is weightage score of the plurality of independent variables with respect to the at least one dependent variable of the second set of subjects,
Xi, Yi . . . are the normalized coordinates of i^{th} patient the first set of subjects for X, Y . . . independent variables,
Xj, Yj . . . are the normalized coordinates of j^{th} patient the second set of subjects for X, Y . . . independent variables.
In an example, the weighted euclidean distance and weighted score between Treatment Patient 1 and Placebo Patient 1, 2, 3, 4 and 5 is

| | Weightage | Treatment | Placebo | Placebo | Placebo | Placebo | Placebo |
|---|---|---|---|---|---|---|---|
| Feature Name | (Ix) | Patient 1 | Patient 1 | Patient 2 | Patient 3 | Patient 4 | Patient 5 |
| Treatment | 19.1% | 40 | 34 | 38 | 44 | 32 | 51 |
| Duration | | | | | | | |
| (Minutes) | | | | | | | |
| Body Mass | 17.2% | 29.8 | 23 | 26.5 | 34.3 | 28.7 | 20.3 |
| Index (Kg/m²) | | | | | | | |
| Protocol | 10.5% | 12 | 12 | 3 | 3 | 0 | 9 |
| Deviations- | | | | | | | |
| Tests / | | | | | | | |
| Assessments / | | | | | | | |
| Procedures | | | | | | | |
| (Occurrences) | | | | | | | |
| Test Score 1 at | 9.2% | 58.6 | 43.9 | 58.6 | 41.7 | 58.6 | NA |
| Baseline Visit | | | | | | | |
| (Score) | | | | | | | |
| Vital Signs- | 7.9% | 110 | 130 | 127 | 131 | 136 | 86 |
| Systolic Blood | | | | | | | |
| Pressure at | | | | | | | |
| Baseline Visit | | | | | | | |
| (mmHg) | | | | | | | |
| Test Score 2 at | 7.7% | 64 | 22 | 55 | 24 | 48 | 26 |
| Baseline Visit | | | | | | | |
| (Score) | | | | | | | |
| Vital Signs- | 7.2% | 37 | 36.8 | 37 | 36.7 | 36.9 | 36.6 |
| Temperature at | | | | | | | |
| Baseline Visit | | | | | | | |
| (° C.) | | | | | | | |
| Test Score 3 at | 5.8% | 92 | 40 | 77 | 51 | 71 | 60 |
| Baseline Visit | | | | | | | |
| (Score) | | | | | | | |
| Weight (Kg) | 4.4% | 83 | 65 | 96.2 | 124.3 | 83.3 | 47 |
| Test Score 4 at | 3.1% | 10 | 7 | 6 | 10 | 10 | 23 |
| Baseline Visit | | | | | | | |
| (Score) | | | | | | | |
| Medical History | 2.9% | 0 | 4 | 1 | 4 | 0 | 4 |
| Surgical and | | | | | | | |
| medical | | | | | | | |
| procedures | | | | | | | |
| (Occurrences) | | | | | | | |
| Age (Years) | 2.7% | 44 | 21 | 35 | 21 | 40 | 37 |
| Medical History- | 2.5% | 2 | 6 | 5 | 2 | 2 | 4 |
| Nervous | | | | | | | |
| system | | | | | | | |
| disorders | | | | | | | |
| (Occurrences) | | | | | | | |

In an embodiment, in order to determine the weighted Euclidean distance, the normalized imputed independent variables are represented in a multi-dimensional space. Herein, at least one dimension of the multi-dimensional space represents at least one independent variable of the subject from the plurality of independent variable. In this regard, each of the plurality of subjects are represented in the multi-dimensional space, wherein the representation of at least one independent variable is based on normalized imputed values for corresponding subject. Notably, the normalized subject values refer to scaled subject values. Moreover, each dimension of the multi-dimensional space represents a variable from the plurality of independent variables.

The server arrangement is configured to determine a placebo effect for each of the first set of subjects undergoing treatment. Optionally, the placebo effect is calculated based on

$${P}_{j} = \frac{{\sum }_{1}^{n} Δ {f}_{i} / {d}_{i}}{{\sum }_{1}^{n} 1 / {d}_{i}} ;$$

Pⱼ is the calculated placebo effect for j^{th} patient in the first set of subjects,
Δfi is the at least one dependent variable for i^{th} patient in the second set of subjects,
n is the total number of patients in the second set of subjects,
di is weighted Euclidean distance between i^{th} patient in the second set of subjects and j^{th} patient in the first set of subjects.
In an example, the placebo effect can be determined based the following data:

| | | Similarity | | |
|---|---|---|---|---|
| | Weighted | Index | | Contribution |
| | Distance | (= 1/ | | to Placebo |
| | From | Weighted | Improvement | Effect of |
| Second set | Treatment | Euclidean | over | Treatment |
| of subjects | Patient 1 | Distance) | Baseline | Patient 1 |
| Placebo | 0.46 | 2.17 | −1 | −0.09 |
| Patient 1 | | | | |
| Placebo | 0.46 | 2.17 | −3 | −0.27 |
| Patient 2 | | | | |
| Placebo | 0.57 | 1.75 | 1 | 0.07 |
| Patient 3 | | | | |
| Placebo | 0.57 | 1.75 | 1 | 0.07 |
| Patient 4 | | | | |
| Placebo | 0.58 | 1.72 | 10 | 0.7 |
| Patient 5 | | | | |
| Placebo | 0.59 | 1.69 | 1 | 0.07 |
| Patient 6 | | | | |
| Placebo | 0.61 | 1.64 | 5 | 0.33 |
| Patient 7 | | | | |
| Placebo | 0.62 | 1.61 | −7 | −0.46 |
| Patient 8 | | | | |
| Placebo | 0.63 | 1.59 | −2 | −0.13 |
| Patient 9 | | | | |
| Placebo | 0.64 | 1.56 | 9 | 0.57 |
| Patient 10 | | | | |
| Placebo | 0.69 | 1.45 | 2 | 0.12 |
| Patient 11 | | | | |
| Placebo | 0.71 | 1.41 | 1 | 0.06 |
| Patient 12 | | | | |
| Placebo | 0.71 | 1.41 | 4 | 0.23 |
| Patient 13 | | | | |
| Placebo | 0.72 | 1.39 | 7 | 0.4 |
| Patient 14 | | | | |
| Placebo | 0.86 | 1.16 | 7 | 0.33 |
| Patient 15 | | | | |
| Total | | 24.47 | | 2.00 |

Based on similarity of treatment patient 1 with all the 15 placebo group patients and placebo (i.e. total) effect of each placebo group patient, the placebo effect in treatment patient 1 is 2.00. This is done for all the patients in the treatment arm.

Optionally, the placebo effect for each of the first set of subjects, his/her similarity (i.e. inverse of the Weighted Euclidean Distance) from second set of subjects is multiplied with the at least one dependent variable for that second set of subjects and normalized with sum of inverse of the Weighted Euclidean Distance from the plurality of dataset from the all the second set of subjects receiving placebo. This gives that the second set of subjects receiving placebo patient's contribution to the Placebo Effect of that Treatment arm patient. Summing up this contribution from each Placebo arm patient yields the Placebo Effect for that Treatment arm patient. Optionally, the treatment effect for that patient is therefore Total Effect score minus the calculated Placebo Effect. In an example, the total effect is the at least one dependent variable value i.e. the improvement over baseline. In the above-mentioned example, if the placebo effect of the subject is 2, whereas the improvement over the baseline for a weight loss patient is 10 i.e. total effect. The treatment effect of the subject patient is 8.

In yet another aspect, an embodiment of the present disclosure provides a computer program product comprising non-transitory computer-readable storage media having computer-readable instructions stored thereon, the computer-readable instructions being executable by a computerized device comprising processing hardware to execute the aforesaid method.

### DETAILED DESCRIPTION OF DRAWINGS

Referring to FIG. 1 is a schematic illustration of a data flow diagram representing a method 100 for determining treatment effect in a clinical trial. At step 102, the method comprises receiving a plurality of datasets corresponding to a plurality of subjects from the database arrangement. At Step 104, the method comprises imputing one or more values in the plurality of datasets to generate an imputed dataset of the plurality of subjects. At step 106, the method comprises normalizing the imputed dataset corresponding to the plurality of independent variable of the plurality of subjects. At step 108, the method comprises determining a plurality of weightage score of the plurality of independent variables with respect to the at least one dependent variable of the second set of subjects. At step 110, the method comprises determining a plurality of similarity score between each of the first set of subjects undergoing treatment to each of the second set of subjects receiving placebo. At step 112, the method comprises determining a placebo effect for each of the first set of subjects undergoing treatment. At step 114, the method comprises determining a treatment effect for each of the first set of subjects undergoing treatment.

It will be appreciated that FIG. 1′ is merely an example, which should not unduly limit the scope of the claims herein. It is to be understood that the specific designation for method 100 determining treatment effect in a clinical trial. It is provided as an example and is not to be construed as limiting the method 100 to specific processes, numbers, types, or arrangements of modules, user devices, servers, sources of input data, and communication networks. A person skilled in the art will recognize many variations, alternatives, and modifications of embodiments of the present disclosure.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system for determining treatment effect in a clinical trial, the system comprising a server arrangement communicably coupled to a database arrangement, wherein the server arrangement is configured to:
(a) receive a plurality of datasets corresponding to a plurality of subjects from the database arrangement, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) impute one or more values in the plurality of datasets to generate an imputed dataset of the plurality of subjects, wherein the imputed dataset comprises of imputed and non-imputed values corresponding to the plurality of independent variables and the at least one dependent variable,
(c) normalize the imputed dataset corresponding to the plurality of independent variable of the plurality of subjects,
(d) determine a plurality of weightage score of the plurality of independent variables with respect to the at least one dependent variable of the second set of subjects,
(e) determine a plurality of similarity score between each of the first set of subjects undergoing treatment to each of the second set of subjects receiving placebo,
(f) determine a placebo effect for each of the first set of subjects undergoing treatment, and
(g) determine a treatment effect for each of the first set of subjects undergoing treatment.

2. A system of claim 1, wherein the treatment effect for each of the first set of subjects undergoing treatment is based on total effect score and minus the placebo effect, wherein the total effect is based on the at least one dependent variables of the subject of the first set of subjects.

3. A system of claim 1, wherein the independent variables comprise of continuous variables and categorical variables.

4. A system of claim 1, wherein the at least one dependent variable is based on improvement over baseline on an index measured at pre-defined frequency during the clinical trial.

5. A system of claim 1, wherein the one or more values of the continuous variables of the independent variable are imputed by a mean of the values of the corresponding independent variable, wherein the one or more values of the categorical variables are imputed by a mode of the values of the corresponding independent variable, wherein one or more values of the at least one dependent variable is imputed by Last Observed Carried Forward (LOCF) method of the corresponding dependent variable.

6. A system of claim 1, wherein the continuous variables of the independent variable are normalized using Min-Max Scaler, and wherein the categorical variables of the independent variable are one-hot-encoded into dummy variables.

7. A system of claim 1, wherein the plurality of weightage score of the plurality of independent variables with respect to the at least one dependent variable of the second set of subjects is based on SHAP importance of XGBoost regressor model.

8. A system of claim 1, wherein the plurality of similarity score between each of the first set of subjects undergoing treatment to each of the second set of subjects receiving placebo is based on an inverse of weighted Euclidean distance, wherein the weighted Euclidean distance is calculated using the plurality of weightage score, the normalized imputed dataset of plurality of independent variables from first set of subjects and the normalized imputed dataset of plurality of independent variables from second set of subjects.

9. A system of claim 8, wherein the weighted Euclidean distance is calculated based on
Dᵢⱼ={square root over ((Iₓ(Xᵢ−Xⱼ))²+(I_{Y}(Yᵢ−Yⱼ))²+ . . . )}
where ‘i’ is the i'^{th} patient in the first set of subjects undergoing treatment and j is the j^{th} patient in the second set of subjects receiving placebo,
Dᵢⱼ is the weighted Euclidean distance between these patients,
Iₓ is weightage score of the plurality of independent variables with respect to the at least one dependent variable of the second set of subjects,
Xi, Yi . . . are the normalized coordinates of i^{th} patient the first set of subjects for X, Y . . . independent variables,
Xj, Yj . . . are the normalized coordinates of j^{th} patient the second set of subjects for X, Y . . . independent variables.

10. A system of claim 1, wherein the placebo effect is calculated based on
$${P}_{j} = \frac{{\sum }_{1}^{n} Δ {f}_{i} / {d}_{i}}{{\sum }_{1}^{n} 1 / {d}_{i}} ;$$
Pₜ is the calculated placebo effect for t^{th} patient in the first set of subjects, Δfi is the at least one dependent variable for i^{th} patient in the second set of subjects,
n is the total number of patients in the second set of subjects,
dᵢ is weighted Euclidean distance between i^{th} patient in the second set of subjects and t^{th} patient in the first set of subjects.

11. A method of determining treatment effect of a clinical trial, wherein the method comprises:
(a) receiving a plurality of datasets corresponding to a plurality of subjects, wherein the plurality of subjects comprises a first set of subjects undergoing treatment and a second set of subjects receiving placebo, and wherein the plurality of datasets comprises of plurality independent variables and at least one dependent variables for the plurality of subjects;
(b) imputing one or more values in the plurality of datasets to generate an imputed dataset of the plurality of subjects, wherein the imputed dataset comprises of imputed and non-imputed values corresponding to the plurality of independent variables and the at least one dependent variable,
(c) normalizing the imputed dataset corresponding to the plurality of independent variable of the plurality of subjects,
(d) determining a plurality of weightage score of the plurality of independent variables with respect to the at least one dependent variable of the second set of subjects,
(e) determining a plurality of similarity scores between each of the first set of subjects undergoing treatment to each of the second set of subjects receiving placebo,
(f) determining a placebo effect for each of the first set of subjects undergoing treatment, and
(g) determining a treatment effect for each of the first set of subjects undergoing treatment.

12. A method of claim 11, wherein the treatment effect for each of the first set of subjects undergoing treatment is based on total effect score and the placebo effect, wherein the total effect is based on the at least one dependent variables of the subject of the first set of subjects.

13. A method of claim 11, wherein the independent variables comprise of continuous variables and categorical variables.

14. A method of claim 11, wherein the at least one dependent variable is based on improvement over baseline on an index measured at pre-defined frequency during the clinical trial.

15. A method of claim 11, wherein the one or more values of the continuous variables of the independent variable are imputed by a mean of the values of the corresponding independent variable, wherein the one or more values of the categorical variables are imputed by a mode of the values of the corresponding independent variable, wherein one or more values of the at least one dependent variables is imputed by Last Observed Carried Forward (LOCF) method of the corresponding dependent variable.

16. A method of claim 11, wherein the continuous variables of the independent variable are normalized using Min-Max Scaler, and wherein the categorical variables of the independent variable are one-hot-encoded into dummy variables.

17. A method of claim 11, wherein the plurality of weightage score of the plurality of independent variables with respect to the at least one dependent variable of the second set of subjects is based on SHAP importance of XGBoost regressor model.

18. A method of claim 11, wherein the plurality of similarity scores between each of the first set of subjects undergoing treatment to each of the second set of subjects receiving placebo is based on an inverse of weighted euclidean distance, wherein the weighted Euclidean distance is calculated using the plurality of weightage score, the normalized imputed dataset of plurality of independent variables from first set of subjects, and the normalized imputed dataset of plurality of independent variables from second set of subjects.

19. A method of claim 18, wherein the weighted Euclidean distance is calculated based on
Dᵢⱼ={square root over ((Iₓ(Xᵢ−Xⱼ))²+(I_{Y}(Yᵢ−Yⱼ))²+ . . . )}
where ‘i’ is the i'^{th} patient in the first set of subjects undergoing treatment and j is the j^{th} patient in the second set of subjects receiving placebo,
Dᵢⱼ is the weighted euclidean distance between these patients,
Iₓ is weightage score of the plurality of independent variables with respect to the at least one dependent variable of the second set of subjects,
Xi, Yi . . . are the normalized coordinates of i^{th} patient the first set of subjects for X, Y . . . independent variables,
Xj, Yj . . . are the normalized coordinates of j^{th} patient the second set of subjects for X, Y . . . independent variables.

20. A method of claim 11, wherein the placebo effect is calculated based on
$${P}_{j} = \frac{{\sum }_{1}^{n} Δ {f}_{i} / {d}_{i}}{{\sum }_{1}^{n} 1 / {d}_{i}} ;$$
Pₜ is the calculated placebo effect for t^{th} patient in the first set of subjects,
Δfi is the at least one dependent variable for i^{th} patient in the second set of subjects,
n is the total number of patients in the second set of subjects,
dᵢ is weighted euclidean distance between i^{th} patient in the second set of subjects and t^{th} patient in the first set of subjects.

21. A computer program product comprising non-transitory computer-readable storage media having computer-readable instructions stored thereon, the computer-readable instructions being executable by a computerized device comprising processing hardware to execute a method of claim 11.
